(19) United States
(12) Patent Application Publication
(10) Pub. No.: US 2016/0166732 A1
(43) Pub. Date: Jun. 16, 2016

(54) SKIN PRINTING AND AUTO-GRAFTING

(71) Applicant: Dermagenesis LLC, Miami, FL (US)

(72) Inventors: David Tumey, Pompano Beach, FL (US); Sandra Berriman, Pompano Beach, FL (US)

(21) Appl. No.: 15/050,508

(22) Filed: Feb. 23, 2016

Related U.S. Application Data
(62) Division of application No. 14/331,314, filed on Jul. 15, 2014.
Application No. 14/331,314 is a continuation-in-part of application No. 14/084,896, filed on Nov. 20, 2013.

(51) Int. Cl. A61L 27/36; A61L 27/38; C12N 5/071; A61L 27/60

(52) U.S. Cl. CPC A61L 27/3691; A61L 27/60; A61L 27/3839; A61L 27/3813; C12N 5/0698; A61L 2430/34; A61L 2420/02; A61L 2400/18; A61L 2300/64; A61L 2300/606; B33Y 10/00

(57) ABSTRACT

A holey substrate now is used for constructing a graft product, such as building an autograft by 3D printing of living cells. When the autograft built atop the holey substrate is implanted, blood vessels and other patient tissues can grow through the holes.

### FIELD OF THE INVENTION

The invention relates to the medical arts, more particularly, to tissue engineering especially tissue engineering in which three-dimensional printing technology is used.

### BACKGROUND OF THE INVENTION

Healing wounds is a complex process of tissue repair and regeneration in response to injury. The healing response in skin wounds attempts to reconstitute a tissue similar to the original damaged one and this is accomplished via the concerted action of numerous skin cell types, collagens, cytokines, growth factors (GF s), chemokines, cell surface and adhesion molecules, as well as multiple extracellular matrix proteins. Autologous split-thickness skin grafting currently represents the most rapid, effective method of reconstructing large skin defects; however, in cases where a significant quantity of harvested graft is required, it represents yet another trauma to an already injured patient.

Some patent literature and academic literature is mentioned as follows, generally in roughly chronological order:
Wille, Jr., “Method for the formation of a histologically-complete skin substitute,” U.S. Pat. No. 5,292,655 issued Mar. 8, 1994;
Bernard, et al., “Process for creating a skin substitute and the resulting skin substitute,” U.S. Pat. No. 5,639,654 issued Jun. 17, 1997;
Bernard et al., “Skin substitute,” U.S. Pat. No. 5,667,961 issued Sep. 16, 1997;
Wille, Jr., “Serum free medium for use in the formation of a histologically complete living human skin substitute,” U.S. Pat. No. 5,686,307 issued Nov. 11, 1997;
Takai et al., “Wound healing composition using squid chitin and fish skin collagen,” U.S. Pat. No. 5,698,228 issued Dec. 16, 1997;
Wille, Jr., “Cell competency solution for use in the formation of a histologically-complete, living, human skin substitute,” U.S. Pat. No. 5,795,781 issued Aug. 18, 1998;
S. Hybbinette et al., “Enzymatic dissociation of keratinocytes from human skin biopsies for in vitro cell propagation,” Exp Dermatol., 1999: February; 8(1):30-8;
Mares-Guia, “Non-immunogenic, biocompatible macromolecular membrane compositions, and methods for making them,” U.S. Pat. No. 6,262,255 issued Jul. 17, 2001;
D. W. Hutmacher, “Scaffold design and fabrication technologies for engineering tissues—state of the art and future perspectives,” J. Biomater. Sci. Polymer Edn, 12:1, 107-124 (2001);
Conrad et al., “Skin substitutes and uses thereof,” US 20020164793 published Nov. 7, 2002;
Ramos et al., “Method for the preparation of immunologically inert amniotic membranes,” US 20040126878 published Jul. 1, 2004;
Conrad et al., “Skin substitutes and uses thereof,” U.S. Pat. No. 6,846,675 issued Jan. 25, 2005;
Conrad et al., “Skin substitutes and uses thereof,” US 20050226853 published Oct. 13, 2005;
S. G. Priya, et al., “Skin Tissue Engineering for Tissue Repair and Regeneration,” Tissue Engineering: Part B, 14:1, 2008, 105-118;
G. S. Schultz, et al., “Interactions between extracellular matrix and growth factors in wound healing,” Wound Rep Reg 17, 153-162 (2009);
Conrad et al., “Skin substitutes and uses thereof,” U.S. Pat. No. 7,541,188 issued Jun. 2, 2009;
Woodroof, “Laser-Perforated Skin Substitute,” US 20090230592 published Sep. 17, 2009;
Woodroof, et al., “Artificial Skin Substitute,” US 20090232878 published Sep. 17, 2009;
Woodroof; “Temporary Skin Substitute comprised of biological compounds of plant and animal origins,” US 20090234305 published Sep. 17, 2009;
Woodroof, et al., “Skin Substitute Manufacturing Method,” US 20100000676 published Jan. 7, 2010;
Woodroof, et al., “Artificial skin substitute,” U.S. Pat. No. 7,815,931 issued Oct. 19, 2010;
Mirua, et al., “Skin Substitute Membrane, Mold, and Method of Evaluating External Preparation for Skin,” US 20110098815 published Apr. 28, 2011;
Israelowitz et al., “Apparatus for the growth of artificial organic items, especially human or animal skin,” US 20110159582 published Jun. 30, 2011;
Guenou, “Methods for Preparing Human Skin Substitutes from Human Pluripotent Stem Cells,” US 20110165130 published Jul. 7, 2011;
Bush et al., “Bioengineered Skin Substitutes,” US 20110171180 published Jul. 14, 2011;
Yoo et al., “Delivery system,” US 20110172611 published Jul. 14, 2011;
M. V. Karaaltin et al., “Adipose Derived Regenerative Cell Therapy for Treating a Diabetic Wound: A Case Report,” Oct. 6, 2011;
Chernokalskaya, et al., “Polymeric Membranes with Human Skin-like Permeability Properties and uses thereof,” US 20110281771 published Nov. 17, 2011;
Miura et al., “Application method of external dermatological medications, evaluating method of the same, application evaluating apparatus, and application evaluating program,” US 20120022472 published Jan. 26, 2012;
D. Rosenblatt, “Researchers aim to ‘print’ human skin,” Feb. 15, 2011, www.cnn.com;
Miura et al., “Skin substitute membrane, mold, and method of evaluating external preparation for skin,” US 20120109300 published May 3, 2012;
R. Kirsner, et al., “Spray-applied cell therapy with human allogeneic fibroblasts and kertinocytes for the treatment of chronic venous leg ulcers: a phase 2, multicentre, double-blind, randomised, placebo-controlled trial,” www.thelancet.com, vol. 380, Sep. 15, 2012;
B. Raelin, “Wake Forest 3D Prints Skin Cells Onto Burn Wounds,” Jul. 19, 2012, www.3dprinter-world.com;
A. Lutz, “Printed Skin Cells Will Change How We Treat Burns Forever”, Aug. 3, 2012, www.businessinsider.com;
Miura et al., “Skin substitute membrane, mold, and method of evaluating external preparation for skin,” U.S. Pat. No. 8,337,554 issued Dec. 25, 2012;
“Printing Skin,” www.medicaldiscoverynews.com/shows/202_printSkin.html, undated;
C. M. Zelen, et al., “A prospective randomised comparative parallel study of amniotic membrane wound graft in the management of diabetic foot ulcers,” International Wound Journal, ISSN 1742-4801, 2013;
H. Kim, et al., “Evaluation of an Amniotic Membrane-Collagen Dermal Substitute in the Management of Full-Thickness Skin Defects in a Pig,” Archives of Plastic Surgery, 2013, 40:1, 11-18;
“SkinPrint: 3D Bio-printed human skin can help burn victims”, May 16, 2013, www.3ders.org;
K. Maxey, “3D Printed, Transplantable Skin in 5 Years?”, May 17, 2013, www.engineering.com;
H. Briggs, “Artificial human ear grown in lab,” Jul. 31, 2013, www.bbc.co.uk;
S. Leckart, “How 3-D Printing Body Parts Will Revolutionize Medicine,” Aug. 6, 2013, www.popsci.com;
Thangapazham et al., “Hair follicle neogenesis,” US 20130209427 published Aug. 15, 20131
T. Lu et al., “Techniques for fabrication and construction of three-dimensional scaffolds for tissue engineering,” Internat'l Journal of Nanomedicine, 2013:8, 337-350.

Although there are a number of reports of skin autografts produced in vitro, they take weeks to generate—which is too long a waiting period for a patient whose wound needs treatment. Quicker production of skin autografts is an unmet need and unsolved problem.

In several studies conducted using amniotic membrane (AM) in both acute and chronic wounds, much of the first round placement is absorbed into the body. In some cases, it takes as many of 3-4 full grafts of AM in order to result in full closure of the wound. Less graft being absorbed into the body so that it is unable to contribute to closing the wound is an unsolved problem.

Another difficult unsolved problem has been that when an undamaged donor area of skin of a patient is harvested and used as an autograft for treating the patient's own wound, the donor site often becomes a non-healing wound.

There are complicated, unsolved problems and unmet needs for better technologies in wound grafting and wound healing.

### SUMMARY OF THE INVENTION

The invention addresses the above-described problems by processing ALL of the harvested skin cells taken from a healthy donor site on the patient with the wound to construct a customized skin graft product to be auto-grafted onto the wound. Production of a customized skin graft preferably is accomplished by operation of a three-dimensional (“3D”) printer, which is supplied with substrate material (preferably a holey substrate) and autologous skin cells and “prints” the supplied skin cells onto an agar plate or other surface.

Advantageously the amount of donor dermal cells needed from non-wound areas of a patient having a wound to be auto-grafted is reduced by using all of the harvested skin cells. A 3D printer is used to construct a wound graft product from the harvested skin cells without wasting any of the harvested skin cells. In a case of an irregularly shaped wound, wastage of harvested skin associated with trimming is avoided. The invention's provision of a skin grafting method that requires only the least amount of precious skin of the donor site to be damaged is highly important given the major functions of skin: acting as a protective barrier from environmental insults including trauma, radiation, harsh environmental conditions and infection, providing thermoregulation (through sweating, vasoconstriction or vasodilation) and controlling fluid loss. This minimization of skin damage provided by the invention, in addition to the ability to continually regenerate the necessary skin until healing is complete, represent major advances in wound care.

A major objective of the invention is to use the patient's own skill cells to re-create a strong, persistent organ replacement solution.

The invention in a preferred embodiment provides a computerized skin printing system, comprising: a quantity of living donor skin cells harvested from a non-wound area of a patient having a to-be-treated wound or tissue defect; a 3D printer that processes the quantity of living donor skin cells harvested from a non-wound area of a patient having the wound or tissue defect, wherein the 3D printer is under control of a controller connected to the 3D printer; an imaging device (such as, e.g., an imaging device that comprises a camera; an imaging device that comprises a video camera; an imaging device that comprises a hand-held device; an imaging device that is movable to be positioned relative to the wound being imaged; etc.); and a computer that performs steps of receiving a set of images (such as, e.g., a set of one wound image; a set of multiple images) taken by the imaging device of the wound or tissue defect and processing the imaged wound or tissue defect into a set of skin-printing instructions that are provided to the controller connected to the 3D printer; such as, e.g., a skin printing system further comprising a sizing grid that is projected onto the wound or tissue defect while the imaging device is being operated; a skin printing system further comprising a monitor connected to the computer; a skin printing system further comprising a keyboard connected to the computer; a skin printing system further comprising at least one syringe pump (such as e.g., a syringe pump that contains the quantity of living donor skin cells harvested from the non-wound area) under control of the controller; a skin printing system further comprising a surface onto which the 3D printer prints a skin product (such as, e.g., a skin printing system wherein the skin product printed onto the surface corresponds to a model generated by the computer from the set of wound images); a skin printing system further comprising a pump, and wherein skin cells in a syringe are pumped by the pump into the three-dimensional printer; a skin printing system wherein the computer digitizes a wound image and models the digitized image into a set of printing instructions; a skin printing system further comprising an agar plate comprising the surface onto which the 3D printer prints the skin product; a skin printing system wherein the 3D printer is supplied with both a quantity of living skin cells from the patient with the imaged wound and a quantity of material not from the patient with the imaged wound (such as, e.g., collagen or another scaffold-building material as the non-patient material); a skin printing system further comprising a digitizer; and other inventive skin printing systems.

In another preferred embodiment, the invention provides an autograft treatment method of a wound of a patient, comprising: preparing the wound to be imaged; imaging the wound to obtain a set of images (such as, e.g., a wound imaging step that comprises photographing the wound); based on the set of images of the wound, modeling (such as, e.g., 3D modeling) a skin graft product, wherein the modeling is performed by a computer, processor, or other machine; harvesting dermal cells from the patient (such as, e.g., from a donor site of the patient; from a wound of the patient); from the harvested dermal cells, preparing a live cell suspension; loading a plate into a 3D printer (such as, e.g., a printer-loading step that comprises loading an agar gel plate onto a platen of the printer); constructing a scaffold onto the plate (such as, e.g., a scaffold-constructing step in which the scaffold is constructed using little or none of the live cell suspension; a scaffold-constructing step that comprises constructing a scaffold of collagen (such as, e.g., bovine collagen (such as, e.g., Bovine Collagen Type 1)); etc.); seeding the scaffold with cells from the live cell suspension, until the modeled skin graft product has been constructed; when the skin graft product has been constructed, removing the skin graft product from the printer and from the plate; and after the removing step, placing the skin graft product in the wound; such as inventive methods wherein in the harvesting step, an amount of dermal cells harvested is in approximately a 1:5 ratio of skin harvested to skin estimated to be needed to treat the wound by conventional skin grafting; inventive methods wherein in the harvesting step, a maximum size is a 4 cm² split-thickness graft using standard dermatome techniques; inventive methods wherein in the harvesting step, an amount of dermal cells harvested is does not exceed a 1:5 ratio of cells harvested to cells estimated to be needed to treat the wound by conventional skin grafting; inventive methods comprising dissociating and culturing the harvested cells in a culture medium (such as methods comprising adding allogeneic fibroblasts and keratinocytes to the culture medium); inventive methods further comprising securing the skin graft product with sutures and covering the skin graft product with a bandage; inventive methods comprising constructing multiple skin graft products for a same wound; inventive methods comprising constructing a first skin graft product and a second skin graft product for a same wound, on different days; a method further comprising printing insulin into the skin graft product being constructed; a method further comprising printing or spraying amniotic membrane into the skin graft product being constructed; and other inventive methods.

In another preferred embodiment, the invention provides a skin graft product constructed from skin cells of a patient having a wound, wherein an amount of patient skin cells is less than the patient skin cells that would be estimated to be needed to treat the wound if only the patient skin cells were used, such as, e.g., an inventive skin graft product consisting of: an amount of patient skin cells which is less than the patient skill cells that would be estimated to be needed to treat the wound by conventional skin grafting if only the patient skin cells were used; and an amount of material other than patient skin cells; an inventive skin graft product wherein the amount of patient skin cells is selected from the group consisting of: about ⅔ what would be estimated to be needed to treat the wound if only the patient skin cells were used; less than ⅔ what would be estimated to be needed to treat the wound if only the patient skin cells were used; less than ½ what would be estimated to be needed to treat the wound if only the patient skin cells were used; less than ⅓ what would be estimated to be needed to treat the wound if only the patient skin cells were used; less than ¼ what would be estimated to be needed to treat the wound if only the patient skin cells were used; and less than ⅕ what would be estimated to be needed to treat the wound if only the patient skin cells were used; an inventive skin graft product wherein the amount of material other than patient skin cells comprises one or more of bovine collagen, growth factors, amniotic membrane and cytokines; and other inventive skin graft products.

In another preferred embodiment, the invention provides a method of treating a patient wound, comprising: constructing a set of custom skin graft products G1 . . . Gn customized to the wound; placing the custom skin graft product G1 onto the wound; and placing the custom skin graft product Gn onto the custom skin graft product Gn−1 already placed on the wound, such as, e.g., an inventive method comprising layering custom skin graft products onto the wound over a period of days; and other inventive methods.

The invention in another preferred embodiment provides a method of avoiding wastage of dermal cells harvested for autografting to treat a wound of a patient, comprising: harvesting a quantity of skin cells from a non-wound site of the patient having the wound; processing all of the harvested quantity of skin cells into an autograft skin product without wasting or discarding any of the harvested quantity of skin cells (such as, e.g., a processing step that comprises three-dimensional printing of an irregular three-dimensional shape); and applying the autograft skin product onto the wound; such as, e.g., inventive methods further comprising meshing the autograft; inventive methods wherein a ratio of surface area of the wound to surface area of a harvest site is about 5 square inches of wound to 1 square inch of harvest site, which is expressed as a Wound/Harvest Areas Ratio of 5:1; inventive methods wherein a Wound/Harvest Areas Ratio is in a range of from 2:1 to 7:1; inventive methods wherein the Wound/Harvest Areas Ratio is in a range of from 5:1 to 7:1; and other inventive methods.

The invention in another preferred embodiment provides an auto-grafting method for treating a wound of a patient, comprising: harvesting a quantity of skin cells from a patient; and auto-grafting onto the wound of the patient the quantity of harvested skin cells, with the quantity of autografted harvested skin cells being substantially equal to the quantity of harvested skin cells (such as, e.g., an auto-grafting step that comprises auto-grafting a three-dimensional irregularly-shaped skin graft product); an auto-grafting method further comprising constructing, via operation of a 3D printer, a skin graft product comprising the quantity of harvested skin cells; and other inventive auto-grafting methods.

In another preferred embodiment, the invention provides a substrate implantable in a patient, comprising: a holey thin, flat substrate layer comprising a plurality of holes, wherein each hole completely penetrates the substrate layer and is of a size bigger than a blood vessel that grows in an area being treated, wherein the hole accommodates the blood vessel growing through the hole in a pattern entering from a first side of the substrate and exiting on a second side of the substrate; such as, e.g., an inventive substrate having a honeycomb shape; an inventive honeycomb-shaped substrate that comprises a plurality of hexagon-shaped holes, wherein a hexagon-shaped hole has a width of about 3 mm; an inventive substrate further comprising biologic matter (such as, e.g., biologic matter comprising dermal cells; biologic matter comprising cells harvested from the patient; etc.) atop the substrate layer; an inventive substrate wherein the substrate layer consists of a resorbable material that a human body resorbs in a period of weeks or months; an inventive substrate wherein the substrate layer has a thickness in a range of about 1-3 microns; etc.

The invention in another preferred embodiment comprises a cell printing method, comprising: printing, performed by a 3D printer stocked with a quantity of living cells, cells in tracks onto a holey substrate (such as, e.g., a holey substrate that is a honeycomb-patterned substrate), wherein the holey substrate is characterized by a plurality of holes; such as, e.g., an inventive cell printing method comprising printing cells in layers (such as, e.g., an inventive cell printing method comprising printing different numbers of layers of cells in different areas of the substrate); an inventive cell printing method comprising printing collagen onto the holey substrate, followed by printing fibroblasts onto the collagen; and other inventive methods.

The invention in another preferred embodiment provides a wound treatment product comprising: a resorbable material shaped as a honeycomb structure, the honeycomb structure being relatively flat; and, a quantity of living cells (such as, e.g., living cells that comprise living skin cells), and optionally one or more selected from the group consisting of (a) collagen, (b) collagen matrix; (c) collagen matrix proteins, and (d) extracellular matrix proteins; atop the honeycomb structure.

Another preferred embodiment of the invention provides a computerized skin printing system, comprising: a quantity of living donor skin cells harvested from a patient having a to-be-treated wound or tissue defect; a three-dimensional printer that processes the quantity of living donor skin cells, wherein the three-dimensional printer is under control of a controller connected to the three-dimensional printer; an imaging device; and, a computer that performs steps of receiving a set of images taken by the imaging device of the wound or tissue defect and processing the imaged wound or tissue defect into a set of skin-printing instructions that are provided to the controller connected to the three-dimensional printer; such as, e.g., an inventive skin printing system further comprising a holey substrate onto which the three-dimensional printer prints a skin product; and other inventive skin printing systems.

The invention in another preferred embodiment provides an autograft treatment method of a wound of a patient, comprising steps of: (1) preparing the wound to be imaged; (2) imaging the wound to obtain a set of images; (3) based on the set of images of the wound, modeling a skin graft product, wherein the modeling is performed by a computer, processor, or other machine; (4) harvesting dermal cells from the patient; (5) from the harvested dermal cells, preparing a live cell suspension; (6) seeding a holey substrate with cells from the live cell suspension, until the modeled skin graft product has been constructed; (7) when the skin graft product has been constructed, removing the skin graft product from the printer; and (8) after the removing step, placing the skin graft product in the wound.

In another preferred embodiment the invention provides an auto-grafting method for treating a wound of a patient, comprising steps of: harvesting a quantity of skin cells from a patient; and, auto-grafting onto the wound of the patient the quantity of harvested skin cells supported by a holey substrate, with the quantity of autografted harvested skin cells being substantially equal to the quantity of harvested skin cells, such as, e.g., an inventive auto-grafting method further comprising, after the auto-grafting step, providing a channel through which a blood vessel grows through the holey substrate wherein the channel-providing is performed by a hole in the holey substrate; an inventive auto-grafting method further comprising constructing, via operation of a three-dimensional printer, a skin graft product comprising the quantity of harvested skin cells on a holey substrate; and other inventive auto-grafting methods.

Also in another preferred embodiment the invention provides a lattice implantable in a patient, comprising: a lattice structure defined by a structural material; and, a plurality of holes, wherein each hole traverses the lattice structure and is of a size bigger than a blood vessel that grows in an area being treated, wherein the hole accommodates the blood vessel growing through the hole in a pattern entering from a first face of the lattice structure and exiting on a second face of the lattice structure; such as, e.g., an inventive lattice comprising a quantity of living cells layered onto the structural material, directly or atop a layer of collagen; and other inventive lattices.

### BRIEF DESCRIPTION OF FIGURES

FIG. 1 is a diagram of a computerized skin printing system in an embodiment of the invention.

FIG. 2 is a diagram of an inventive method of producing an inventive autograft product, in an embodiment of the invention.

FIG. 3 is a diagram of steps in an inventive autologous grafting method.

FIG. 4 is a top view of a holey substrate according to an exemplary embodiment of the invention.

### DETAILED DESCRIPTION OF PREFERRED EMBODIMENT OF THE INVENTION

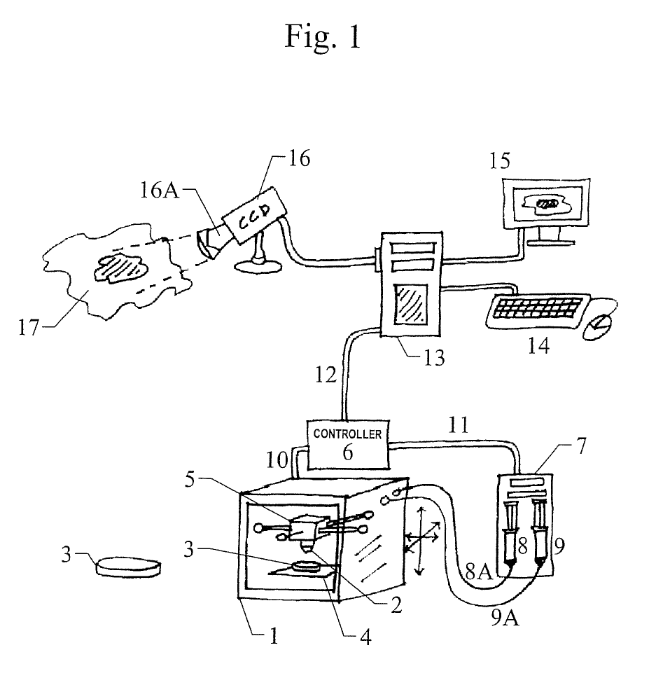

In a dermal autograft that comprises a quantity of harvested patient dermal cells, the invention advantageously minimizes the quantity of harvested patient dermal cells that are needed for an autograft to cover a particular wound. For such minimization, harvested patient dermal cells (preferably ALL of the harvested patient dermal cells) are used in combination with a quantity of material which is NOT harvested patient dermal cells, to construct a dermal autograft product to be applied to a wound. Preferred construction methods for use in the invention are, e.g., a layering method performed by a 3D printer (such as, e.g., 3D printer 1 in FIG. 1); a method in which a computerized skin printing system is used (such as a computerized skin printing system of FIG. 1, see Example 1 herein); etc.

A preferred example of material which is NOT harvested patient dermal cells and which is useable in the invention is collagen, such as, e.g., Bovine Collagen Type I; Collagen IV; etc. As to Collagen IV, see, e.g., M. Paulsson, “Basement Membrane Proteins: Structure, Assembly, and Cellular Interactions,” Critical Reviews in Biochemistry and Molecular Biology, 27(½): 93-127 (1992).

The inventive methodology preferably is used to fabricate then print skin tissue using much smaller areas of donor skin (such as, e.g., no larger than 4 cm² split-thickness grafts harvested using standard dermatome techniques) compared to conventional methodology, or even to avoid using healthy donor skin and instead use donor tissue from the wound itself. The invention's provision of the ability to use such smaller areas of donor skin corresponds to a significant reduction in skin injury and subsequently less opportunity for transformation into a chronic wound or other sequelae common to donor sites. Advantageously, the invention provides an improved ratio of wound area to donor site (such as a 5:1 ratio of wound area to donor site; a 6:1 ratio of wound area to donor site; a 7:1 ratio of wound area to donor site; etc.) compared to a grafting methodology having a 1:1 up to 3:1 ratio of wound area to donor site for a mesh graft. Advantageously, the invention is useable for a relatively small area of donor site to cover relatively much wound site, such as, e.g., being able to cover 5-7 times, or more, of the donor site.

A preferred methodology of combining harvested patient dermal cells and other material which is NOT harvested patient dermal cells is for cells from the respective donor site and non-donor sources to be processed until ready for loading into a set of dispensers in a 3D printer, and the 3D printer is used to perform a printing process by which the patient dermal cells and other materials are printed into a unitary graft product.

In one example of obtaining the patient dermal cells, small split thickness skin grafts are created and epidermal cells harvested, after which the heterogeneous mixture of cell types comprising mainly fibroblasts and keratinocytes is dissociated and cultured using standard cell culture techniques. Preferably, to stimulate rapid proliferation, allogeneic fibroblasts and keratinocytes are added to culture media along with a cocktail including appropriate growth factors.

In a preferred example of a printing process, autologous cells which have been incubated with allogeneic fibroblasts and keratinocytes are printed onto a bovine collagen matrix in the size, shape, and depth of the patient's particular wound. In a most preferred example, collagen is printed first, then skin cells are layered onto the collagen. Preferably the collagen matrix is fortified with growth factors, amniotic membrane, and specific cytokines which serve as an active extracellular matrix (ECM) and basement membrane structure. Such procedures are preferred in order to set in motion a process by which the partially autologous skin graft will mimic the architecture of the patient's own tissue.

Following preparation of the wound bed, a skin structure produced according to the invention is transplanted into the analogous structure of the wound.

An advantage of the invention is to use the patient's own skill cells to re-create a strong, persistent organ replacement solution.

Additionally, the time in which the replacement product is produced is much faster than the weeks needed to generate skin autografts produced in vitro using conventional methodology. The current state of the science has not reported manipulating cell proliferation at the rate needed for a 3-7 day growing phase. By contrast, advantageously, 3D cell printing according to the invention using an enhanced cell proliferation method with a mixture of cell types, ECM proteins, growth factors, and cytokines greatly reduces the time for regeneration of an adequate skin graft suitable for transplantation and healing.

Unlike skin substitutes such as the dermal matrices Alloderm (human cadaveric), Strattice, or Integra (porcine sources) which are cost prohibitive and can be immunoreactive, the invention advantageously is used to recreate or regenerate a patient's own skin, in the shape and depth analogous to the injury. The resulting graft is less expensive compared to the mentioned products and has a better chance to “take”. Addition of allogeneic cells bolster and enhance proliferation of the patient's own fibroblasts and keratinocytes, and provide a source of constituents such as extracellular matrix and growth factors.

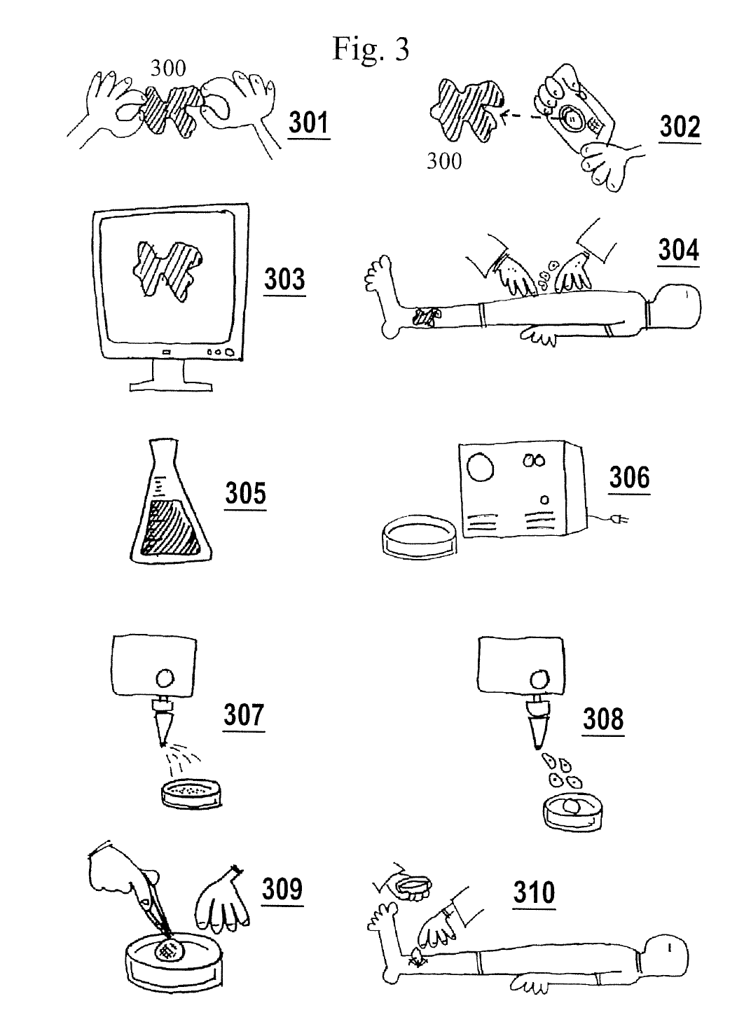

As may be further appreciated with reference to FIG. 3, an example of an inventive skin printing process is step-wise as follows:

1) Preparing 301 the wound 300 (e.g., NPWT—to manage exudate, reduce/eliminate infection, create vascularized granular bed of tissue).

2) Photographing 302 the wound 300.

3) Automatically modeling 303 the to-be-produced graft in 3D from the wound photo.

4) Obtaining 304 dermal cells from donor site (estimating a ratio, such as estimating a 1:5 ratio). Examples of the donor site include, e.g., a wounded area of the patient; a non-wounded area of the patient. A non-wounded area of the patient is conventionally recognized as where to obtain a skin graft. The present inventors have determined that a wounded area of the patient also is useable as a donor site for dermal cells to be used in the invention.

5) Preparing 305 a live cell suspension using the obtained dermal cells.

6) Loading 306 a plate (such as an agar plate) into a 3D printer (such as by loading an agar plate onto a platen of a 3D skin printer).

7) Physically rendering 307 an acellular dermal matrix (ADM) scaffold with collagen (such as pre-processed Bovin Collagen Type I). Preferably the scaffold is a holey substrate.

8) Seeding 308 the ADM scaffold with live cells processed from the autologous graft obtained in step 4 of this Example (step 304 in FIG. 3). Note, ADM may contain allogeneic fibroblasts. This step is also accomplished by “printing” the cells onto the ADM.

9) Removing 309 printed skin from the 3D printer and agar gel plate.

10) Performing a step 310 of placing the printed skin in the wound 300, securing (such as, e.g., securing with sutures, securing with medical cyanoacrylates, etc.) and covering with a suitable bandage.

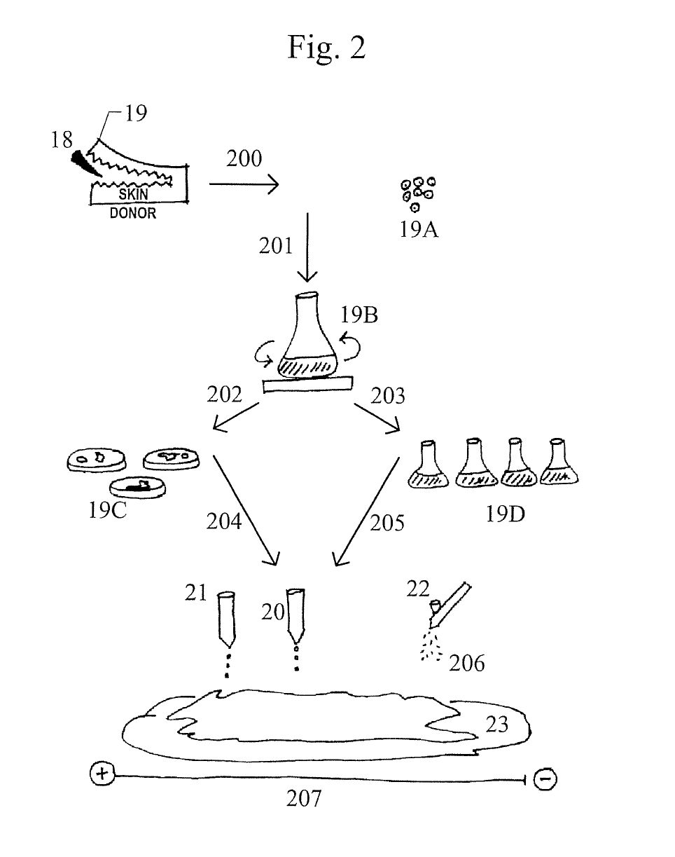

An inventive method of producing an inventive autograft product also can be appreciated with reference to FIG. 2. Surgical instrument 18 is used to separate epidermis 19 from skin at a donor site preferably of a same patient who has wound 17 (FIG. 1).

Separated epidermis 19 is processed 200 by enzymatic cell separation to produce separated dermal cells 19A which are dissolved 201 to produce a dermal cell solution or suspension 19B.

Dermal cell solution or suspension 19B is cultured 202 onto plates to provide plated dermal cells 19C and/or is split 203 into dermal cell solutions 19D (such as 70% confluency).

Cultured dermal cells 19C and dermal cell solutions 19D are harvested 204, 205 to be transferred to 3D printer cell dispensers such as dispenser 20.

Examples of contents of 3D printer cell dispenser 20 are, e.g., autologous fibroblasts, keratinocytes, ECM proteins, growth factors (GF s), cytokines. Examples of contents of 3D printer cell dispenser 21 are, e.g., GF, insulin, PDGF, eNOS. Examples of contents of 3D printer cell dispenser 22 are lyophyllized amniotic membrane.

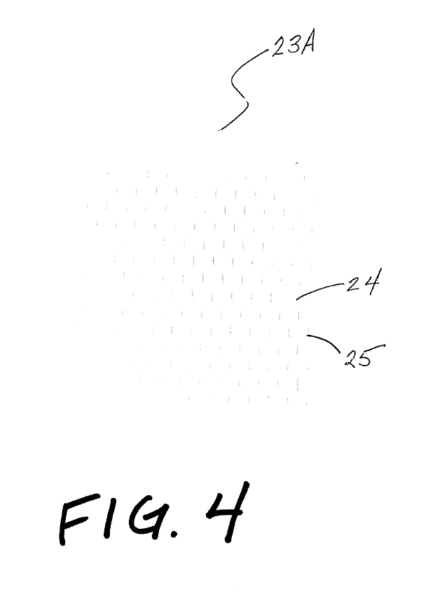

A 3D printer (such as 3D printer 1 of FIG. 1) prints 206 the contents of the dispensers 20, 21, 22 onto a substrate 23 (preferably a holey substrate) to produce a cultured graft preferably comprising bovine collagen, media, growth factors (GF s), etc. A holey substrate is preferred for substrate 23 because blood vessels will be able to grow through the holes and the blood vessels will be able to supply the autograft. Advantages of providing holes in the substrate 23 include, e.g., that patient's tissues will grow into the graft and/or that cells, growth factors, cytokines, etc. can grow into the graft. Holey substrate 23A (FIG. 4) comprises a plurality of holes 24 each hole having a size bigger than a blood vessel that is expected to grow in a vicinity of an autograft. Holes 24 are shown as hexagonally-shaped in FIG. 4 for purpose of illustration but are not required to be hexagonal and may be of other geometric shapes or an irregular shape. Holes 24 are defined by absence of solid material 25. An example of producing a holey substrate 23A is production via a 3D printer that prints using a starting material that is resorbable by the human body such as, e.g., bioresorbable glass materials, etc.

Optionally an electrical field 207 is applied in a region of substrate 23 (preferably a holey substrate) during printing 206.

It will be appreciated that printing 206 from dispensers 20, 21, 22 is not required to be performed simultaneously and that printing 206 may be performed in various sequences.

An example of harvesting grafts is to harvest a first graft at 7 days (from when the epidermis was removed from the donor site), and to maintain other grafts unharvested for a period of time until needed through final closure.

The invention may be further appreciated with reference to the following examples, without the invention being limited thereto.

### Example 1

In one inventive example, as may be appreciated with reference to FIG. 1, an inventive computerized skin printing system comprises a 3D printer 1. Preferably the 3D printer 1 is cooled or temperature-controlled. An example of a 3D printer 1 is a 3D printer capable of printing living cells. The 3D printer 1 comprises at least one dispenser head 2 from which emerges cells that are being printed onto a surface 3 (such as, e.g., an agar plate) which is accommodated on a platen 4 within the 3D printer. The dispenser head 2 is attached to print head 5 which is positionable in (x, y, z) dimensions, which positioning is controlled by controller 6. Controller 6 also controls a syringe pumping system 7.

Syringe pumping system 7 comprises syringe 8 in which is contained skin cells harvested from the patient for whom the auto-graft product is being made and syringe 9 in which is contained material which does NOT include the patient's skin cells, such as, e.g., bovine collagen; allogeneic skin cells; etc. System 7 optionally comprises static mixers. Syringes 8, 9 supply the 3D printer 1 via tubes 8A, 9A respectively. Components used by the 3D printer to print an auto-graft skin product are pumped from syringes 8, 9 to the dispenser head 2.

Controller 6 is electrically connected by electrical connection 10 to the 3D printer 1 and by electrical connection 11 to the pumping system 7.

Controller 6 is electrically connected via data line 12 to a computer 13. As an example of computer 13 is a computer comprising a digitizer, the computer having software loaded thereon such as, e.g., software that digitizes an image of a wound and models the defect for printing; software that digitizes an image of a wound and automatically detects wound boundaries and models the defect for printing; etc. In some embodiments, wound boundaries are manually detected. Computer 13 receives human operator input via an input device 14 which in FIG. 1 is illustrated as a keyboard but is not necessarily limited to a keyboard. A human operator reviews output from computer 13 on a monitor 15.

Components illustrated separately in FIG. 1, such as, e.g., input device 14 and monitor 15, are not necessarily required to be separate physical structures and can be integral with each other. Also, in FIG. 1, cables or connecting lines that are illustrated are not necessarily required in all embodiments to be physical structures and in some embodiments a wireless connection is provided.

Computer 13 is connected to an imaging device 16 such as, e.g., a camera. Preferably imaging device 16 delivers video images to computer 13. Imaging device 16 is positionable to image a wound on a living patient, such as, e.g., being positionable via a stable structure such as an articulated arm, tripod, cart or frame. Imaging device comprises a component 16A (such as, e.g., a lens) which in operation is positioned in a direction of a wound or other tissue defect 17. Preferably a sizing guide (such as, e.g., a sizing grid) is provided in a region of the wound 17 (such as, e.g., a laser grid for sizing) while the imaging device 16 is imaging the wound 17. Preferably a laser sizing grid is projected onto and/or near the wound 17 to provide data for sizing the wound. In another embodiment, graticulated markers are positioned proximate the wound to provide sizing information to the imaging device 16.

Preferably computer 13 performs steps of receiving a set of images taken by the imaging device 16 of the wound or tissue defect 17 and processing the imaged wound or tissue defect into a set of skin-printing instructions that are provided to the controller 6 connected to the 3D printer 1.

The system of FIG. 1 is useable to process a quantity of living donor skin cells harvested from a non-wound area of a patient having the wound or tissue defect 17.

### Example 2

Application of the dissociated cells and other agents by the 3D printer, specifically, the configuration of the cell dispenser/applicator/syringe/air-brush, is dependent upon the type and depth of the wound. The number of “layers” or “passes” the cell dispenser must take with each agent applied to the collagen matrix in this Example is at least one layer.

This approach of layering the patient's own fibroblasts, keratinocytes, etc., with commercially available amniotic membrane, growth factors, etc., is used to manipulate the healing process through wound supplementation with agents that are natural contributors to the wound healing process and specifically crucial for each particular wound type.

### Example 3

Examples of techniques are as follows.

### Example 3.1

Following harvest of the donor site, individual cells of the epidermal layer are dissociated from the dermis. Dissociation of skin cells is accomplished by traditional trypsin: EDTA methods which is a preferable method for isolating keratinocytes from human skin. Human serum, bovine serum albumin, serum fibronectin, type IV collagen, and laminin added to traditional cell culture media provide support to the fibroblasts and keratinocytes. These basement membrane protein constituents form the layers of the extracellular matrix on which these epidermal and dermal cells grow. They are present in every tissue of the human body. They are always in close apposition to cells and it is well known that they not only provide structural support in the form of an organized scaffold, but they also provide functional input to influence cellular behavior such as adhesion, shape, migration, proliferation, and differentiation. Disassociated cells are incubated and continually shaken in cell culture flasks at 37° C. Cells are sub-cultured prior to confluency and allowed either to continue to proliferate in dissociated cell suspension flasks, plated on collagen plates to continue growth, or plated via the skin printer onto bovine collagen substrates.

### Example 3.2

In this Example, a bovine collagen matrix is augmented with growth factors such as Platelet-Derived Growth Factor (PDGF), epidermal Nitric Oxide Synthase (eNOS), Vascular Endothelial Growth Factor (VEGF), and Tumor Necrosis Factor Beta (TNF-beta). Low-dose insulin is added to also promote cell growth and proliferation. Insulin is a powerful growth factor that has been used in animal and human clinical trials of wound healing. Insulin has been used as a topical agent to accelerate the rate of wound healing and the proportion of wounds that heal in diabetic animals and in humans. Treatment with insulin also increased expression of eNOS, VEGF, and SDF-1alpha in wounded skin. Rezvani conducted an RCT in diabetic foot wounds to evaluate topical insulin on healing in 45 patients. The mean rate of healing was 46.09 mm²/day in the treatment group, and 32.24 mm²/day in the control group (p=0.03). These data suggest that insulin can improve wound healing and may be beneficial when used in an in vitro model to increase cell proliferation and would enhance cell proliferation into the collagen matrix.

### Example 3.3

3-4 days following the first application of autologous cells, and as the allogeneic cells and matrix begin to form obvious healthy epithelial tissue, lyophilized amniotic membrane (AM) is sprayed (such as from a modified airbrush-like apparatus (preferably associated with the print head of the 3D printer) onto the cell-seeded bovine collagen. There is a notable body of evidence to suggest that freeze-dried, powdered amniotic membrane promotes rapid healing and enhances the “take” rate of grafts. AM also inhibits natural inflammatory reactions which contribute to healthy tissue adhesion and structural development. There is evidence to suggest that combined with an electrical field, the application of AM will enhance cell migration and angiogenesis to cells located in the center-most region of the graft bed.

### Example 3.4

Continual layers of the cultured material are printed onto collagen plates until desired thickness is achieved. Amount of cells wanted in each layer, number of times the printer must create layers for the skin graft, intervals between applications, and types and amounts of growth factors and other ECM proteins to be added are factors.

### Example 4

Multiple copies of the autograft are printed. (In this example, multiple copies are printed. It will be appreciated that in other cases due to limited donor site material there will only be enough to print one copy.) The first is transplanted to the primary wound within 5-7 days. During the 5-7 days preparation period, negative pressure wound therapy with or without simultaneous irrigation (e.g., saline) is applied to prepare the wound bed for graft acceptance as well as reduce bacterial load. Negative pressure therapy is known to induce angiogenesis and this increase in blood flow and the resultant delivery of nutrients not only to the wound bed but to the newly placed engineered craft is critical to its survival and success.

As was described hereinabove in the Background with respect to several studies conducted using amniotic membrane (AM) in both acute and chronic wounds, much of the first round placement was absorbed into the body. In some cases, it took as many of 3-4 full grafts of AM in order to result in full closure of the wound when using that conventional technology. By contrast, with skin printing according to the invention, a much thicker and partially autologous engineered graft that more closely approximates natural human skin is provided. A thicker, partially autologous engineered graft has improved probability of survival and ability to make active contributions to recruiting the active mechanisms of healing. Meanwhile, in practicing the invention, the additional skin grafts continue to mature and if necessary, are useable as the final step to closure. In the alternative, the graft copies could be stored in a tissue bank for later use by the same patient if, for example, additional surgical revisions were anticipated.

While the invention has been described in terms of a preferred embodiment, those skilled in the art will recognize that the invention can be practiced with modification within the spirit and scope of the appended claims.

1-17. (canceled)

18. An autograft treatment method of a wound of a patient, comprising:
(1) preparing the wound to be imaged;
(2) imaging the wound to obtain a set of images;
(3) based on the set of images of the wound, modeling a skin graft product, wherein the modeling is performed by a computer, processor, or other machine;
(4) harvesting dermal cells from the patient;
(5) from the harvested dermal cells, preparing a live cell suspension;
(6) seeding a holey substrate with cells from the live cell suspension, until the modeled skin graft product has been constructed;
(7) when the skin graft product has been constructed, removing the skin graft product from the printer;
(8) after the removing step, placing the skin graft product in the wound.

19. An auto-grafting method for treating a wound of a patient, comprising:
harvesting a quantity of skin cells from a patient;
auto-grafting onto the wound of the patient the quantity of harvested skin cells supported by a holey substrate, with the quantity of autografted harvested skin cells being substantially equal to the quantity of harvested skin cells.

20. The auto-grafting method of claim 19, further comprising, after the auto-grafting step, providing a channel through which a blood vessel grows through the holey substrate wherein the channel-providing is performed by a hole in the holey substrate.

21. The auto-grafting method of claim 19, further comprising constructing, via operation of a three-dimensional printer, a skin graft product comprising the quantity of harvested skin cells on a holey substrate.

22-23. (canceled)

24. The auto-grafting method of claim 18, wherein in the seeding step, the holey substrate comprises a holey thin, flat substrate layer comprising a plurality of holes, wherein each hole completely penetrates the substrate layer and is of a size bigger than a blood vessel that grows in an area being treated, wherein the hole accommodates the blood vessel growing through the hole in a pattern entering from a first side of the substrate and exiting on a second side of the substrate.

25. The auto-grating method of claim 18, wherein in the seeding step, the holey substrate has a honeycomb shape.

26. The auto-grafting method of claim 25, wherein the honeycomb-shaped substrate comprises a plurality of hexagon-shaped holes, wherein a hexagon-shaped hole has a width of about 3 mm.

27. The auto-grafting method of claim 18, wherein in the seeding step, the holey substrate consists of a resorbable material that a human body resorbs in a period of weeks or months.

28. The auto-grafting method of claim 18, wherein in the seeding step, the holey substrate has a thickness in a range of about 1-3 microns.
